(19) United States
(12) Patent Application Publication
(10) Pub. No.: US 2022/0240854 A1
(43) Pub. Date: Aug. 4, 2022

(54) Communication Device and System for Communicating Exposure to Disease

(71) Applicant: David W. Hornibrook, Tigard, OR (US)

(72) Inventor: David W. Hornibrook, Tigard, OR (US)

(21) Appl. No.: 17/467,133

(22) Filed: Sep. 3, 2021

Related U.S. Application Data
(60) Provisional application No. 63/156,273, filed on Mar. 3, 2021.
(60) Provisional application No. 63/143,192, filed on Jan. 29, 2021.

(51) Int. Cl. A61B 5/00; A41D 13/11; H04W 4/029; H04W 4/38

(52) U.S. Cl. CPC A61B 5/6803; A41D 13/11; A61B 5/0022; H04W 4/029; H04W 4/38; A61B 5/742

(57) ABSTRACT

Systems and methods are provided herein to detect a presence of a target substance and disseminate a notice of the target substance.

### PRIORITY

This application claims priority to U.S. Provisional Application No. 63/156,273, filed Mar. 3, 2021, and also claims priority to U.S. Provisional Application No. 63/143,192, filed Jan. 29, 2021, each of which is incorporated by reference in its entirety herein.

### BACKGROUND

The virus SARS-CoV-2 that causes COVID-19 has gripped the world in a global pandemic of epic proportions. Unfortunately, it is not the first pandemic the world has suffered. Each time, the population, in an attempt to curb the spread of disease, isolates at home, socially distances, wears masks and protective gear, and/or regularly gets tested for the virus or substance causing the disease.

Once a person experiences symptoms, the governmental systems and health organizations may attempt to trace the contacts of the infected individual to identify other potential carriers of the disease. In this way, the continued spread of the disease may be curtailed by limiting the carriers of the disease and reduce contact with the un-infected population. However, such contact tracing is difficult when symptoms may appear days after the event that caused the infection of the infected person. The person may also be contagious after the exposure event, but before expressing symptoms of the disease. The person may therefore not be aware of their harm to others, and may therefore not remember or track their actions or contacts sufficiently close to make contact tracing useful after symptoms have been experienced and/or confirmation of viral infection has occurred.

Detection systems and tests exist for many viruses or other substances of interest that are known to be harmful to people. However, many of these systems require invasive testing or sample collection. Even for those that can detect the presence of a substance by contact or exposure to an environment, such systems provide only a point confirmation of the detected substance. There is no communication system or dissemination of the information that can be useful to others that are not directly performing or controlling the detection test or system.

### SUMMARY

Exemplary embodiments described herein include systems and methods for detecting the presence of a substance of interest and communicating the detection of the substance to a remote location. Embodiments may also include geolocation methods in order to identify a location when the substance of interest is detected. Embodiments may also include distribution notices and other tracing features in which people and/or devices detected in proximity to the identified location when the substance of interest was detected that may have occurred or do occur in temporal proximity (either before and/or after the detection of the substance of interest) to the detection of the substance of interest may also be detected and/or determined.

Exemplary embodiments may include a notification system in which a detection event may provide notifications or warnings to one or more devices and/or people.

### DRAWINGS

FIG. 1 illustrates and exemplary system according to embodiments of the invention.

FIG. 2 illustrates a rapid response detection system in the form of a mask in use by a user.

FIG. 3 illustrates an exemplary rapid response detection system having a dissemination system.

FIG. 4 illustrates an exemplary rapid response detection system and dissemination system according to embodiments described herein.

### DESCRIPTION

The following detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention. It should be understood that the drawings are diagrammatic and schematic representations of exemplary embodiments of the invention, and are not limiting of the present invention nor are they necessarily drawn to scale.

Exemplary embodiments include a mask that will detect the virus SARS-CoV-2 (and variants thereof) responsible for COVID-19, activate the detection and communication system(s), transmit an alert to wearer's cell phone, determine a location, date, and time of the positive detection event, and transmit information to a data collection agency. Exemplary embodiments may also include a phone application that may receive the alert from the mask, determine the GPS (or other location), time in place, time of day, and combinations thereof. The phone application may provide an alert to the user and/or send the information to another location, such as a data collection agency. The data collection agency may provide alerts to local governments and coordinate efforts to reduce cluster breakouts by tracking location hits in similar areas and anticipating contact tracing in real time.

Exemplary embodiments may permit the mask to continue to send alerts until it is reset, providing a way to track the wearer and alert people through their phones that the mask has been exposed. Exemplary embodiments may also include receivers for detecting the alert from the mask and/or phone application to be aware of a positive detection event of the SARS-CoV-2 virus.

Exemplary embodiments may include a molecule that binds with the SARS-CoV-2 virus in a lattice or matrix to form a way to reveal the presence of the SARS-CoV-2 virus, determine the amount of exposure and convey the information to a phone which would then convey the information in real time to a data collection agency along with time, date, and location.

Although embodiments of the invention may be described and illustrated herein in terms of a mask and dissemination system for use with COVID-19, it should be understood that embodiments of this invention are not so limited, but are additionally applicable to other detection and dissemination systems for identifying potential exposure to other diseases or conditions. For example, systems and methods can be used to identify exposure to undesirable substances, allergens, diseases, contaminants, etc. As another example, the system may be incorporated into other forms and not just a mask. For example, it may be in the detector that is attached to clothing, put at desired locations within a facility, or other interfaces to provide notice and disseminate alerts of a positive detection event.

Exemplary embodiments of the systems and methods described herein may include or be configured to work with a rapid response detection system.

Rapid response detection systems preferably include a system and method for detecting the presence of the substance of interest, such as the virus that causes a disease. The rapid response detection system preferably is exposed to an environment in which the target substance resides. The rapid response detection system may be configured to retrieve, detect, or otherwise provide an indication that the target substance is present in the environment to which the rapid response detection system is exposed. In an exemplary embodiment, the rapid response detection system does not require a physical sample collection, such as swabbing, blood draws, wiping, or other active collection steps. The rapid response detection system may include a passive exposure surface, material, volume, or other interface that may detect the presence of the substance of interest through passive contact with the passive contact with the rapid response detection system, or part thereof. The rapid response detection system may also include a self-contained, non-invasive, passive, sampling interface that can be used without training, electronics, supplemental sampling equipment or reagents.

Exemplary systems and methods for detecting the presence of the virus responsible for COVID-19 (SARS-2 CoV-2) are provided herein or otherwise known to a person of skill in the art. For example, U.S. Provisional Patent Application No. 63/000,977, incorporated by reference in its entirety herein, teaches the use of molecularly imprinted polymers for the rapid detection of the virus SARS-CoV-2 responsible for COVID-19. However, the testing shown and described for incorporating on a mask that is used to detect the exhaled breath of the wearer. Exemplary configurations shown and described herein may be configured to detect the presence of the substance of interest in the general environment in which the system for detecting the presence of a substance is positioned and/or the inhaled breath of a wearer of the system for detecting the presence of a substance of interest. Exemplary systems and methods for detecting the presence of the virus are described in the article “Companies develop COVID-19 Detecting face mask” published at canhealth.com/2020/06/17/companies-develop-covid-19-detecting-face-mask/, incorporated in its entirety herein.

Exemplary systems and methods for detecting the presence of a substance of interest, such as SARS-CoV-2, may include a membrane that is configured to indicate the presence of the substance of interest through a color coded response to the rapid test. Other rapid response detection systems may also be used. The system and method for detecting the presence of a substance may also include an interface for monitoring and/or detecting the color coded response. For example, the system and method may include an optical sensor for receiving and/or detecting a color of the membrane to determine whether an indication of the presence of the substance of interest has occurred through a color transition or through identification of a specific color or range of color hues indicating the presence of the substance.

Exemplary systems and methods for detecting the presence of the virus or substance of interest may include a substrate layer comprising a lattice or matrix embedded with an array of docking molecules that the virus would bind to. For example, the substrate may include a molecule that can selectively bind to the spike protein of the SARS-CoV-2 virus. An exemplary molecule may be or include 6-(4-Methylpiperidin-1-yl) sulfonyl-1-azatricyclo[6.6.1.04,12] docdeca-4,6,8(12)-trien-2-one. The binding molecule may be similar to that part of a ligand used in a vaccine. Binding the virus to the docking molecules in the array would prevent or reduce the virus from entering the respiratory system upon inhalation, thus reducing the spread of the disease. The binding within the substrate may also activate the system circuitry established by the lattice or matrix, thereby activating the dissemination system, such as the application of the user's phone as described herein.

Another way to use the molecule would be to suspend in a colloid solution enough molecules to determine exposure. For example, the colloid solution may cause the molecule and SARS virus to precipitate onto a collection layer that would determine parts per million.

Exemplary embodiments include a composition that is configured to detect the presence of the virus or substance of interest. In an exemplary embedment, the detection may be indicated by the emission of light. For example, a protein-based device may be configured to recognize specific molecules on the surface of the virus or substance of interest. The protein-based device may bind to the surface molecules, then emit light through a biochemical reaction. Exemplary embodiments of a modular and tunable protein biosensor is described in the article “De Novo Design of Modular and Tunable Protein Biosensors”, Quijano-Rubio, et al. published at pubmed.gov PMD:33503651 DOI:10.1038/s41586-21-03258-z, incorporated in its entirety herein.

Exemplary embodiments of the system for detection and communication of a substance of interest comprises a layer of biosensors that emit light upon exposure to the substance of interest. The system for detection and communication of a substance of interest may also include an optical sensor that is configured to sense lumens or brightness of the biosensor and determine an amount of exposure.

In an exemplary embodiment, the exterior of the mask may be light blocking in order to reduce interference from ambient light to detect light from the biosensor. In an exemplary embodiment, the interior and/or exterior layer may permit the passage of the virus and the biosensor and light detector may be within the outer layers of the mask. The mask may trap the virus within the mask through one or more other layers. The detection and communication system may include electronics in order to detect the light from the biosensor (such as the light detector) and/or communicate the detection to a remote or separate electronic device, such as a phone.

In an exemplary embodiment, the biosensor may be of a specific wavelength that can be distinctively detected. In an exemplary embodiment, even if environmental light is detected, the light sensor may be configured to detect the light from the biosensor and determine a difference from a threshold (ground) level detected to determine the emission of light from the biosensor above or separate from the light detected from the environment.

In an exemplary embodiment, the biosensor may be used as the indicator to show the presence of the detected substance of interest. The system for detection may include a transparent or translucent portion to permit the light from the biosensor to show through the mask. In an exemplary embodiment, the light sensor may be detected through this same transparent or translucent portion or another similar transparent or translucent portion.

In an exemplary embodiment, a material can be incorporated in the form of a mask to be worn by the wearer on his face. The fabric color may be select to assist with the detection, or reflection, or absorption of the light from the biosensor to assist in the detection of the light from the biosensor.

Exemplary embodiments of the substrate are breathable such that a user can inhale and exhale through the substrate. The substrate and/or mask in which the substrate is incorporated according to embodiments described herein may be configured to filter particulates greater than a desired size. Exemplary embodiments of the substrate and/or mask in which the substrate is incorporated are durable enough to clean and reuse. Exemplary embodiments may be configured to remove the virus and/or reset the array. For example, the system may be exposed to ultraviolet light, infrared radiation, or freezing or reduced temperatures in order to deactivate the transmissivity of the virus within the substrate.

In an exemplary embodiment, the rapid response detection system may include a circuit. The circuit may include a processor, memory, a power source, and combinations thereof. The processor may be a computer chip. The processor may be configured to detect the activation of the substrate and/or color indication and/or other positive detection event from the rapid response detection system. The processor may be configured to reduce false positives by assessing other environmental or input conditions. The processor may include security measures to reduce hacking and/or the generation of the detection alert as described herein.

Exemplary embodiments of the rapid response detection system may include a power source. The power source may be, for example, a silver oxide battery or rechargeable nicad battery in order to power a signal to the dissemination system.

In an exemplary embodiment the rapid response detection system may be incorporated into a mask configured to detect and test the inhaled breath of a wearer. In an exemplary embodiment, the mask may provide an outward indicator of a detection event of a substance of interest on an outside of the mask. For example, a color coded response could be displayed on the outside of the mask so it could be detected at airports, train, bus stations, in classrooms and/or work places. In an exemplary embodiment, the mask may provide an inward indicator of a detection event of a substance of interest on an inside of the mask. For example, the mask may include a layer inside the mask that could serve to alert the wearer if they were shedding the virus through exhalation. This inside layer may be coupled to the alert system and/or dissemination system as described herein.

In an exemplary embodiment, the mask may comprise a thin breathable layer, i.e.: mylar with laser drilled holes and nano pathways interconnected in a grid or lattice pattern. Intersection points would have a molecule that the SARS-CoV-19 virus would dock with completing the circuit and alerting the computer processor to the exposure.

In an exemplary embodiment, the system may include machine readable code stored in memory on a wearable or other interface of the system for detecting the presence of a substance of interest that when executed by a processor of the electronic device is configured to provide the methods described herein. The methods may include identifying the detection of the substance of interest, communicating the presence of the substance of interest to an electronic device, identifying a location of the system at the time of detection, identify a time of detection, and combinations thereof. In an exemplary embodiment, depending on the rapid response detection system used, the system may be configured to count a number of virus hits on a substrate grid (such as a matrix or lattice), compare the amount to a predetermined subset to determine exposure over a pre-set time period. If the exposure density meets or exceeds a dangerous level (such as a predetermined threshold), the system activates an alert and sends the information to the wearers phone. In an exemplary embodiment, the system may transmit an alert until a data collection agency (or receiving device such as the user's phone application) verifies reception and verifies by sending a reset code.

In an exemplary embodiment, the density number can be calibrated from the phone application.

In an exemplary embodiment, the system may monitor power and prompt for battery replacement or recharging in the event the power level falls below a threshold.

Exemplary embodiments may include a communication connection between the system for detecting the presence of a substance of interest and an electronic device. In an exemplary embodiment the electronic device may be a smart phone of the wearer. The electronic device may be other communication devices, such as laptop, tablet, smart watch, bill pay interface, pager, etc. The system and method for detecting the presence of a substance may communicate wirelessly to the system and method for communicating the detection of a substance of interest.

Exemplary embodiments of the systems and methods described herein may include a dissemination system for communicating the detection of the substance of interest. The dissemination system may be configured to provide alerts to one or more users. In an exemplary embodiment, the dissemination system may also include a communication interface, a clock, a location detection system, and combinations thereof.

In an exemplary embodiment, the dissemination system may include a communication interface. The communication interface may provide an alert by providing an indication on a screen, an audible response, tactile or motion response, visual response, or combinations thereof. For example, the dissemination system may include a user interface that may be displayed on an electronic device. The electronic device may be, for example, a mobile phone, smart watch, display screen, smart tv, tablet, electronic reader, laptop, computer, car interface, etc. The dissemination system may be configured to buzz, vibrate, provide a text message, e-mail message, pop up message, or other indication that the exposure has occurred or that a positive detection event has occurred.

The dissemination system may include an electronic device having a communication interface. The communication interface may be configured to send and receive messages from the dissemination system. For example, the communication interface may include Wifi, Bluetooth, RFID, and other communication interfaces for transmitting information to and from the dissemination system. Exemplary embodiments of the communication interface may be configured to transmit the confirmed detection of a substance of interest, a time of detection, a location of the detection, the identity of other detection device within a proximity and/or temporal period from the time of detection and/or location of detection, or combinations thereof. The communication interface may communicate the transmitted information to a remote server.

In an exemplary embodiment, the dissemination system may include a location detection system. For example, the electronic device may include a Global Positioning System (or GPS) interface for communicating with and/or detecting the satellites of the GPS grid for identifying a location of the electronic device. Other location detection systems may also be used, such as those for other geographies and locations. The location detection system may be, for example, a detection of a communication interface and the associated or known location of that detected communication interface. For example, the location detection system may detect the presence of one or more cellular network signal towers. The location detection system may thereafter be configured to determine a location or location area based on the detected cellular network signals.

In an exemplary embodiment, the dissemination system may include a timing device, such as a clock. The clock may be used to record a time that a detection event occurs.

In an exemplary embodiment, the system may include a system and method for communicating the detection of the substance of interest from the system for detecting the presence of a substance of interest. The system for communicating may include machine readable code stored in memory on the electronic device that when executed by a processor of the electronic device is configured to provide the methods described herein. The methods may include receiving a communication from the system for detecting the substance of interest, communicating the presence of the substance of interest to one or more other electronic devices, identify a location of the system at the time of detection, identify a time of detection, and combinations thereof.

In an exemplary embodiment, the system and methods described herein may include a face mask in which the rapid response detection system is incorporated. The face mask may be configured with the rapid response detection system to test the inhaled breath of a user with an indicator. The face mask with the rapid response detection system is saved to a phone application stored on an electronic device of the user. The phone application is configured to alert the user of the face mask in real time if a detection event occurring through the rapid response detection system.

In an exemplary embodiment, the phone application may also be configured with or in communication with a GPS (or other location detection system) to obtain a location of the user at the time of a detection event and send the location and the occurrence of a detection event to a determined destination. The determined destination may be, for example, a data collection agency, such as a local tracking facility, to identify “hot spots” and suggest quarantine to make sure asymptomatic people are not spreading the virus. Exemplary embodiments may be configured to provide text messages to the data collection agency in real time using a universal number like 911.

Exemplary embodiments of the system may also include recognition systems. For example, in the case in which the rapid response detection system provides an indication of a detection event on an outward surface visible to outside observers, the system may include a recognition system for identifying the presence of such indication. In an exemplary embodiment, the system may include optical sensors, such as a camera. The system may include image recognition software configured to detect the presence of the head of a wearer and/or the rapid response detection system, such as a mask. Through facial (or other) recognition technology, the presence of the detection event may be determined using color, fluorescence, ultraviolet, or other signature. For example, the rapid response detection system may be configured to provide an outward indication of the presence of a positive detection event. The system may include recognition systems configured to detect and receive the indication from the rapid response detection system. In an exemplary embodiment, the rapid response detection system may be configured to broadcast a positive detection event such as through a Bluetooth, RFID, or other signal. The recognition system may be configured to detect the broadcast signal from the rapid response detection system. The recognition system may include a communication interface similar to that described herein with respect to the dissemination system. For example, the recognition system may communicate through a network to provide notices to one or more people in the area of the detection event recognized by the recognition system.

In an exemplary embodiment, the recognition system may be employed within a location. The location may be in a restaurant, store, travel station, airplane, classroom, etc. The recognition system may be configured to observe the population through the location and detect the presence of the rapid response detection system and a corresponding positive detection event from a rapid response detection system. The recognition system may be configured to provide notices to the establishment of the location so that cleaning protocols may be implemented, quarantine notices provided, personnel alerted, etc.

Exemplary embodiments of a detection and communication system described herein may include any combination of a rapid response detection system, dissemination system, communication interface, location detection system, clock, recognition system, and combinations thereof. For example components may be duplicated, separated, removed, added, integrated, or otherwise recombined in any combination in order to achieve the objectives of the instant disclosure.

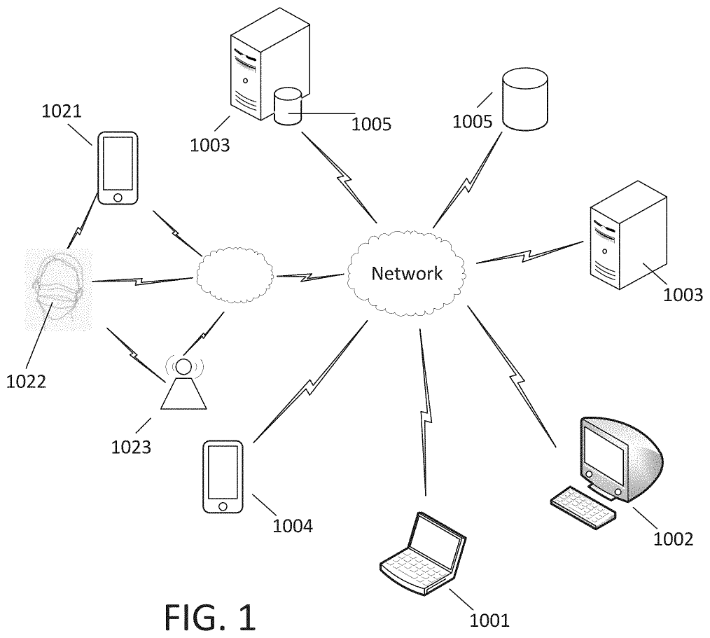

FIG. 1 illustrates exemplary system including a rapid response detection system and dissemination system according to embodiments described herein. Exemplary embodiments of the rapid response detection system described herein may include a mask 1022 in communication with an electronic device 1021. The dissemination system may include the electronic device 1021. The electronic device may be a computer. As used herein, the term computer(s) and/or electronic device(s) are intended to be broadly interpreted to include a variety of systems and devices including personal computers 1002, laptop computers 1001, mainframe computers, servers 1003, set top boxes, digital versatile disc (DVD) players, mobile phone 1004, tablet, smart watch, smart displays, televisions, and the like. A computer can include, for example, processors, memory components for storing data (e.g., read only memory (ROM) and/or random access memory (RAM)), other storage devices, various input/output communication devices and/or modules for network interface capabilities, etc. For example, the system may include one or more processing units including memory, a processor(s), an analog-to-digital converter(s) (A/D), a plurality of software routines that may be stored as non-transitory, machine readable instruction on the memory(ies) and executed by the processor(s) to perform the processes described herein. The processing unit may be based on a variety of commercially available platforms such as a personal computer, a workstation a laptop, a tablet, a mobile electronic device, or may be based on a custom platform that uses application-specific integrated circuits (ASICs) and other custom circuitry to carry out the processes described herein. Additionally, the processing unit may be coupled to one or more input/output (I/O) devices that enable a user to interface to the system. By way of example only, the processing unit may receive user inputs via a keyboard, touchscreen, mouse, scanner, button, or any other data input device and may provide graphical displays to the user via a display unit, which may be, for example, a conventional video monitor. The system may also include one or more large area networks, and/or local networks for communicating data from one or more different components of the system. The one or more electronic devices may therefore input a user interface for displaying information to a user and/or one or more input devices for receiving information from a user. The system may receive and/or display the information after communication to or from the rapid response detection system and/or dissemination system 1022, one or more electronic devices 1001, 1002, 1004, one or more remote server(s) 1003, one or more remote databases 1005, receiver 1024, or any combination thereof.

As illustrated, the rapid response detection system 1022 may be configured to detect a detection event that positively identifies the presence of a substance of interest. The substance of interest may be a virus. The rapid response detection system 1022 may communicate directly or indirectly to an electronic device 1021. The electronic device 1021 may receive information from the rapid response detection system and/or may detect a condition from the rapid response detection system. The electronic device 1021 may determine a location and/or a time of the detection event. The electronic device 1021 may communicate directly or indirectly to other electronic devices such as servers 1003, databases 1005, mobile phones 1004, laptops 1001, computers 1002, pagers, or other electronic devices. Exemplary embodiments of the system permit the electronic device 1021 to send and/or receive information from other electronic devices to provide warnings or notices of positive detection events and/or related data. Exemplary embodiments of the systems described herein may include a recognition system 1023 configured to detect directly or indirectly the rapid response detection system 1022 and receive and/or determine information of a positive detection event. The recognition system 1023 may also communicate to other electronic devices.

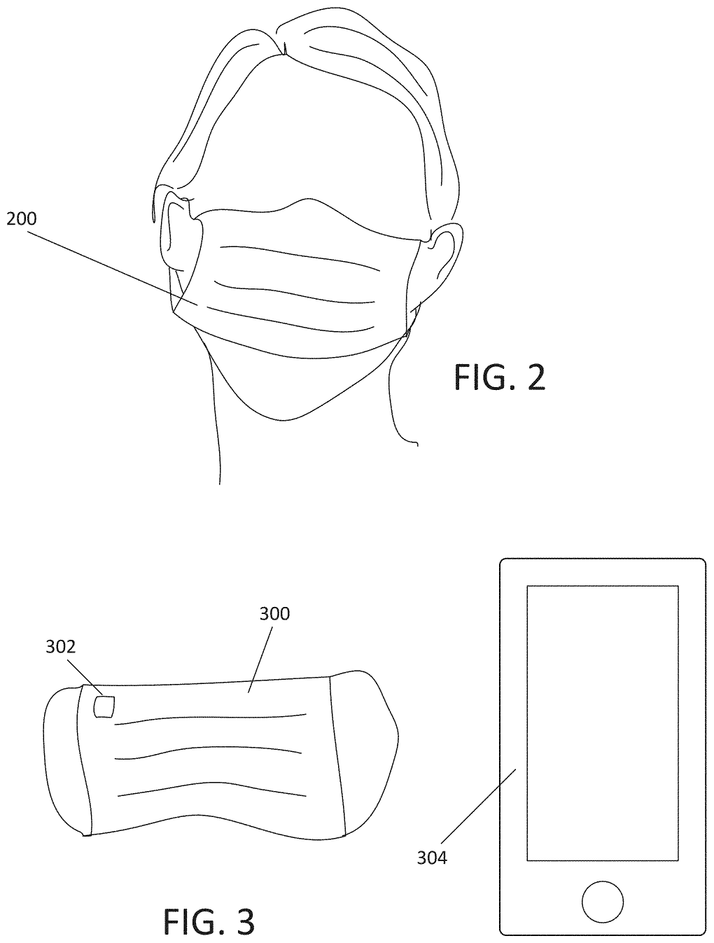

FIG. 2 illustrates a rapid response detection system 200 in the form of a mask in use by a user. The rapid response detection system 200 may include any combination of rapid response embodiments described herein, and/or any combination of dissemination embodiments described herein. As illustrated, the rapid response detection system 200 may be in the form of a mask configured to be positioned over a wearer's face and nose. The mask may be in any form, such as a generally rectangular piece (or other shape) configured to cover the nose and mouth of a wearer and secured to the face of the wearer. The mask may be secured through the use of loops configured to extend around a wearer's ears, straps configured to extend around a user's head, elastic bands, wraps, gators, and any combination thereof. As described in detail herein, the rapid response detection system 200 may be configured to detect a detection event that positively identifies the presence of a substance of interest. The substance of interest may be a virus.

FIG. 3 illustrates an exemplary rapid response detection system 300 having a dissemination system 302. In an exemplary embodiment, the dissemination system 302 may comprise electronic hardware and/or software configured to transmit a signal in response to a positive detection event of the rapid response detection system. In an exemplary embodiment, the dissemination system 302 may communicate directly or indirectly to an electronic device of a user 304. The dissemination system 302 may include a transmitter and/or receiver. The dissemination system 302 may be configured to receive the positive detection event and/or other signal or indication to disseminate a signal indicating a positive detection event. The dissemination system 302 may include a transmitter configured to communicate with the electronic device 304 or another receiver as described herein. The transmitter may be configured to continuously and/or intermittently broadcast a signal indicating a positive detection event.

The electronic device 304 may receive information from the rapid response detection system 302, such as from the dissemination system 302 and/or may detect a condition from the rapid response detection system. The electronic device 302 may comprise hardware and software to achieve the functions described herein. For example, the electronic device may include an application stored in memory and executed by a processor. The application may be configured to run in the background of the electronic device. The application may be configured to continuously and/or intermittently detect the signal from the dissemination system 302. The application may be configured to provide a notice to the user, such as through a display on the screen, sending a text or other communication message, vibrating, making noise, or otherwise providing an immediate alert to the user.

In an exemplary embodiment, the electronic device may include a communications interface to receive the signal transmitted from the dissemination system 302. The electronic device 304 may also include hardware and/or software to determine a location and/or a time of the detection event. The electronic device 302 may be included as part of the dissemination system and may be configured to communicate directly or indirectly to other electronic devices such as receivers configured to provide alerts to other people in an area, or other electronic devices as described herein. The electronic device, such as through the application, may be configured to send information to a remote server and/or database to store information relating to the positive detection event. The information may include the location, date, time, etc.

Exemplary embodiments described herein may also permit additional information and/or electronic device to receive the alert and/or communicate through the system. For example, once a positive detection event is made, the alert may be generally broadcast to other electronic device in proximity. The other electronic device may also have an application running thereon that is configured to communicate to the remote server and/or to the electronic device of the user using the rapid response detection system. The other electronic device may then communicate information to the remote server and/or database to indicate a proximity to a positive detection event in order to be contact traced. The other electronic device may also provide a notice to the respective users of the other electronic device that a positive detection event has occurred in proximity to the device.

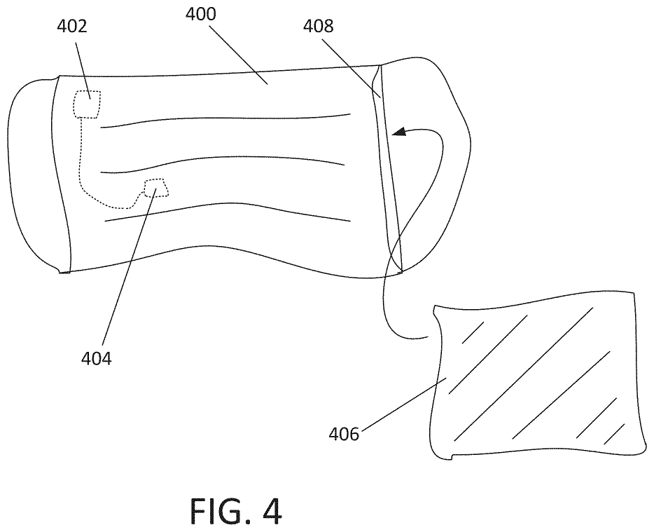

FIG. 4 illustrates an exemplary rapid response detection system and dissemination system according to embodiments described herein. In an exemplary embodiment the system 400 may include a detection system and dissemination system 402. The dissemination system may be as described herein, such as in reference to FIG. 3. The dissemination system 402 may therefore be configured to receive an indication of a positive detection event and then provide a communication of the event away from the mask and/or to the user.

The dissemination system 402, in some embodiments, in addition to or alternatively to the electronic transmission of a signal, the dissemination system may comprise an indicator. For example, the dissemination system 402 may be a color coded indicator to alert a user visually inspecting the mask that a positive detection event has occurred. The indicator may be a light or other visual effect. The indicator may also be a sound or other audial signal. The indicator may be a tactile signal, such as received by the user through vibration.

As illustrated in FIG. 4, the rapid response detection system may include an insert 406 configured to be positioned within the mask, such as within a pocket 408. The mask may be configured to receive the insert and enclose the insert therein. In an exemplary embodiment, the insert may comprise a passive exposure surface configured to be exposed to the environment and/or to the inhaled breath of the user. The passive exposure surface through exposure to the environment may encounter the SARS-CoV-2 virus, or variants thereof. The passive exposure surface may be configured, such as described in any of the embodiments described herein, to detect a positive detection event—the presence of the substance of interest. Exemplary embodiments described herein permit different configurations in which the system may detect a positive detection event. For example, the passive detection surface may be configured to emit light, pass electrical signals, change color, etc.

As illustrated, the system 400 includes a detector for detecting the indication of a positive detection event of the insert 406. Because the indicator of the positive event from the passive detection surface may vary, the detector may also vary and correspond to the indicator of the passive detection surface. For example, if the passive detection surface is configured to emit light, the detector may be configured to detect light; if the passive detection surface is configured to change colors, the detector may be a camera and/or light combination configured to detect a color change and/or detect a specific color; and/or if the passive detection surface is configured to transmit an electrical signal, then the detector may be an electrical connection.

As described herein, the indicator, detector, transmitter, and other combinations of the rapid response detection system and dissemination system may be positioned on the interior of the mask, such as within the pocket 408, may be on an exterior surface of the mask (either outward to the environment or inward toward a user in an in use configuration), or may have components in some combination thereof.

Exemplary embodiments described herein include a rapid response detection system including a mask having: a passive exposure surface configured to detect a presence of a target substance and create an indication of a positive detection event when the target substance is detected; a detector to receive the indication of the positive detection event; and a transmitter configured to transmit a signal in response to the positive detection event.

The rapid response system may include a software application stored in transitory machine readable medium on an electronic device that when executed by a processor of the electronic device is configured to receive the signal transmitted by the transmitter in response to the positive detection event and provide an indication thereof.

The rapid response system may include the indication of a positive detection event through a light transmission, and the detector is a light detector.

The rapid response system may include the indication of a positive detection event through a change in color, and the detector detects the change in color.

The rapid response system may include the indication of the positive detection event through a message displayed on a display of the electronic device.

The rapid response system may include the software application configured to determine a date, a time, and a location of the electronic device when the positive detection event is detected or received by the software application, and the software application is configured to communicate through the electronic device to a remote server the date, time, and location of the electronic device.

Exemplary embodiments described herein include a method for detecting and disseminating a detection event of a target substance, comprising: providing a mask having a passive exposure surface configured to detect a presence of a target substance and create an indication of a positive detection event when the target substance is detected, a detector to receive the indication of the positive detection event, and a transmitter configured to transmit a signal in response to the positive detection event; exposing the passive exposure surface to the environment or to an inhaled or exhaled breath of a wearer; using the passive exposure surface to determine a presence of the target substance; and providing an alert through the transmitter.

The rapid response and dissemination method may include providing the alert by sending through the transmitter the signal; and receiving the signal using an electronic device, and providing an indication of the received signal on the electronic device.

The rapid response and dissemination method may include the indication of the received signal comprising providing a message on a screen of the electronic device.

The rapid response and dissemination method may include transmitting from the electronic device to a remote server another signal related to the positive detection event.

Exemplary embodiments of the system described herein can be based in software and/or hardware. While some specific embodiments of the invention have been shown the invention is not to be limited to these embodiments. For example, most functions performed by electronic hardware components may be duplicated by software emulation. Thus, a software program written to accomplish those same functions may emulate the functionality of the hardware components in input-output circuitry. The invention is to be understood as not limited by the specific embodiments described herein, but only by scope of the appended claims.

Although embodiments of this invention have been fully described with reference to the accompanying drawings, it is to be noted that various changes and modifications will become apparent to those skilled in the art. Such changes and modifications are to be understood as being included within the scope of embodiments of this invention as defined by the appended claims. Specifically, exemplary components are described herein. Any combination of these components may be used in any combination. For example, any component, feature, step or part may be integrated, separated, sub-divided, removed, duplicated, added, or used in any combination and remain within the scope of the present disclosure. Embodiments are exemplary only, and provide an illustrative combination of features, but are not limited thereto.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

1. A rapid response detection system, comprising:
a mask having:
a passive exposure surface configured to detect a presence of a target substance and create an indication of a positive detection event when the target substance is detected;
a detector to receive the indication of the positive detection event; and
a transmitter configured transmit a signal in response to the positive detection event.

2. The rapid response system of claim 1, further comprising a software application stored in transitory machine readable medium on an electronic device that when executed by a processor of the electronic device is configured to receive the signal transmitted by the transmitter in response to the positive detection event and provide an indication thereof.

3. The rapid response system of claim 2, wherein the indication of a positive detection event is through a light transmission, and the detector is a light detector.

4. The rapid response system of claim 2, wherein the indication of appositive detection event is through a change in color, and the detector detects the change in color.

5. The rapid response system of claim 2, where the indication of the positive detection event is through a message displayed on a display of the electronic device.

6. The rapid response system of claim 5, wherein the software application is configured to determine a date, a time, and a location of the electronic device when the positive detection event is detected or received by the software application, and the software application is configured to communicate through the electronic device to a remote server the date, time, and location of the electronic device.

7. A method for detecting and disseminating a detection event of a target substance, comprising:
providing a mask having a passive exposure surface configured to detect a presence of a target substance and create an indication of a positive detection event when the target substance is detected, a detector to receive the indication of the positive detection event, and a transmitter configured to transmit a signal in response to the positive detection event;
exposing the passive exposure surface to the environment or to an inhaled or exhaled breath of a wearer;
using the passive exposure surface to determine a presence of the target substance;
providing an alert through the transmitter.

8. The method of claim 7, wherein the providing the alert comprises sending through the transmitter the signal; and receiving the signal using an electronic device, and providing an indication of the received signal on the electronic device.

9. The method of claim 8, wherein the indication of the received signal comprises providing a message on a screen of the electronic device.

10. The method of claim 9, further comprising transmitting from the electronic device to a remote server another signal related to the positive detection event.
